(19) United States
(12) Patent Application Publication
(10) Pub. No.: US 2015/0190268 A1
(43) Pub. Date: Jul. 9, 2015

(54) AMBULATORY NASAL DEVICE

(71) Applicant: Georges Creps, Montainville (FR)

(72) Inventor: Georges Creps, Montainville (FR)

(21) Appl. No.: 14/576,961

(22) Filed: Dec. 19, 2014

(30) Foreign Application Priority Data
Jan. 3, 2014 (FR) 1450023

(51) Int. Cl. A61F 5/08

(52) U.S. Cl. CPC A61F 5/08

(57) ABSTRACT

This ambulatory nasal device for respiratory assistance comprises at least an intra-nasal structure of expansion (2) intended to be inserted in a nostril, the structure of expansion having a pyramidal form with an annular base (3) and comprising an arch (4) extending from the afore mentioned base (3).

The present invention relates to a device making possible to improve the passage, by nasal way, of the air inspired or expired by a person presenting a particular, obstructive anatomical configuration nasal ways and partitions, this obstruction reducing ventilation in the absence of a mechanical assistance to the opening of the muco-cartilaginous nasal respiratory tracts.

This mechanical assistance is traditionally carried out on the level of the nostrils by assistance systems with their opening.

Certain systems comprise a wing coming to position in each nostril, plated on the mucous membrane. The wings are connected between them by an arch which serves as spacer of the two wings. Such an external arch is however ugly and not very effective in the maintenance in place of the system.

Other systems comprise a vertical tube interns intended to be inserted in each nostril. The tubes are connected between them by an external subnasal spacer. Some of these systems integrate therapeutic reserves of substances.

It was noted that these systems are uncomfortable and ugly. Moreover, they do not eliminate the risk of a dislodgment or a disengagement of the system, in particular during the sleep. In addition, these systems do not enable to obtain a measured and widened anatomical distribution of the support forces to exert, in a homothetic way, on the level of the muco-cartilaginous zones of the nostrils as well as on the level of the muco-cartilaginous zones of the higher nasal air routes. Finally, these systems are likely to generate a physiological secretory blocking of the mucous membranes due to the intimate contact of their structure with the nasal mucous membrane.

It was in addition proposed to use ambulatory devices based on the use of small balloons intended each one to be inserted in a nostril and presenting an end equipped with an opening and an openwork wall. One can refer to document FR1 200 102 which describes such a device.

Although advantageous in many connections, such a device has limited efficiency in the respect of secretory flow, even if it is of however unquestionable interest as an aemostatic support, respecting the passage of the air inspired or expired in a nasal postoperative context.

The device according to the invention enables to overcome these disadvantages.

Thus, an aim of the invention is to provide an ambulatory nasal device of respiratory assistance, including at least a structure of expansion intra-nasal intended to be inserted in a nostril, in which the structure of expansion presents a pyramidal form with an annular base and comprises at least an arch extending starting from the afore mentioned base.

The structures of expansion are intended to be inserted in a receiving site, in particular the intranasal site and the nasal fossae.

The annular base of the structures of expansion, turned towards outside when they are inserted in a nostril, is open in order to support the opening of the nostril. It was noted that such a device including a structure of pyramidal expansion including an arch and an annular base respects the physiology of the nasal mucous membranes, by avoiding blockings of secretions due to the activity of the physiological functions of the nasal mucous membrane.

According to a characteristic, the device is at least partly made out of a material elastically deformable with memory of form.

Thus pyramidal structures of expansion, when they are introduced into the nostrils, by compressive deformation, mould by a homothetic extension, in the research of the initial form, the anatomical reliefs met. These reliefs have a favorable anatomy retentive of the pyramidal structures of expansion avoiding their dislodgment in particular during the sleep.

It will be noted that the device is advantageously carried out by moulding of a plastic and that the degree of elasticity of the device is selected so as to obtain a homothetic pressure measured on the mucous fabrics on which the device rests.

In an embodiment, each structure of expansion comprises a main arch of triangular general form, a main ring connected to the base of the main arch and at least a branch connected to the main arch and said ring.

Furthermore, an intermediate ring can be connected to the main arch and laid out between the top of the main arch and the annular base.

In all the cases, whatever the number of arches and rings, such a structure is naturally adapted to the respect of the anatomical areas on which it is based, by respecting the physiology of the other anatomical areas.

Advantageously, for certain therapeutic applications, the top of the structure of expansion comprises an end spacer including at least a finger-shaped appendix intended to come into position in the area of the triangular cartilage or in a general way in anatomical areas far away from the nasal fossae.

As the rest of the structure of pyramidal expansion, the finger-shaped appendices get a selective support on the mucous membrane to avoid the blocking of secretions. In other words, the supports remain localized while getting a mechanical assistance ready to maintain open the muco-cartilaginous nasal respiratory tracts.

The device can comprise in addition at least an in board tank.

Such tanks can present various forms, for example tubular. They can be fixed in the middle of the structures of expansion and are in particular intended to receive diffusing therapeutic substances for various clinical applications.

It will be noted in this respect that the constitution of the structures of expansion which comprise in particular an arch and an annular base is particularly adapted to leave free the surfaces of the mucous membranes which profit consequently from the diffused substances.

In an embodiment, the device comprises a pair of intra-nasal structures of expansion connected by a spacer.

In this respect a stabilizing spacer can be used, visually discrete, comfortable and transparent. This spacer is intended to be positioned under the external nasal partition and is in particular intended to facilitate the insertion and the withdrawal of the device.

According to another characteristic of the device, each structure of expansion intranasal comprises a transverse wall located on the way of air flow.

For example, the transverse wall is solid or perforated. Advantageously, the transverse wall is made from a material permeable to air.

For example, the permeable material is resorbable. Other goals, characteristics and advantages will appear in the following description, only given to as a non restrictive example, and made in reference to the annexed drawings on which:

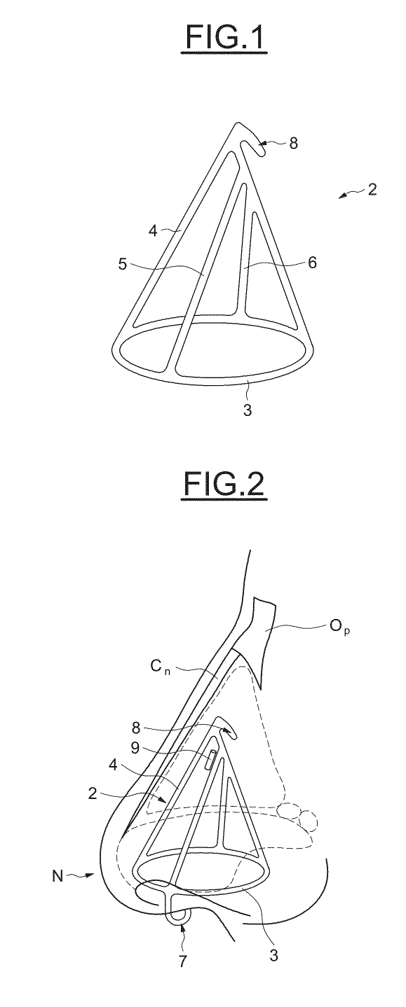

FIG. 1 is a general view of an ambulatory nasal device according to an embodiment of the invention;

FIG. 2 is a general side view of the ambulatory nasal device according to an embodiment of the invention, placed in a nostril;

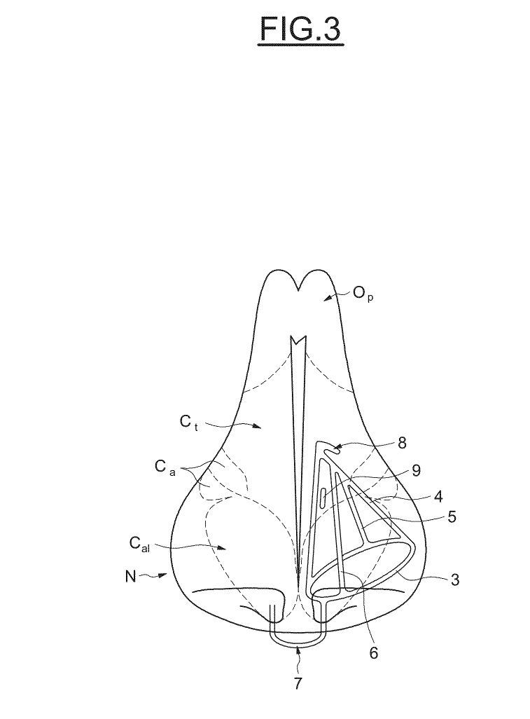

FIG. 3 shows a front view of the device of FIG. 2, installed in a left nostril;

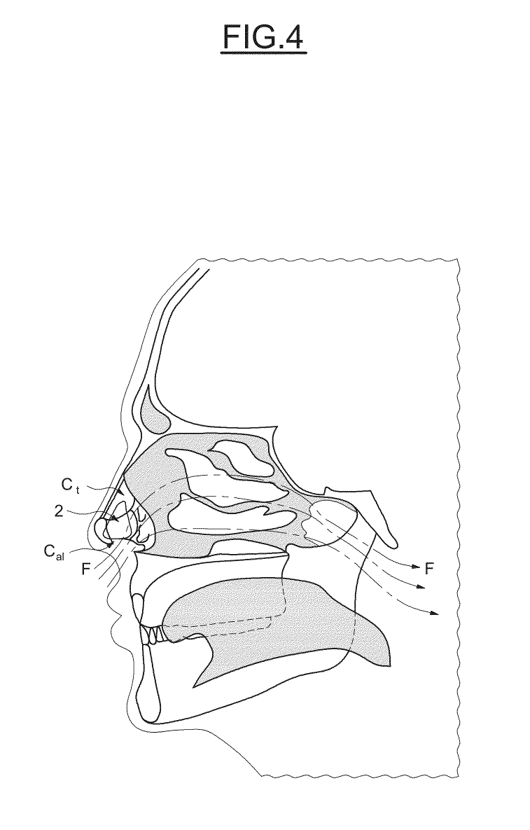

FIG. 4 represents the direction of the air inspired from the wing cartilage towards the triangular cartilage then towards the nasal fossae;

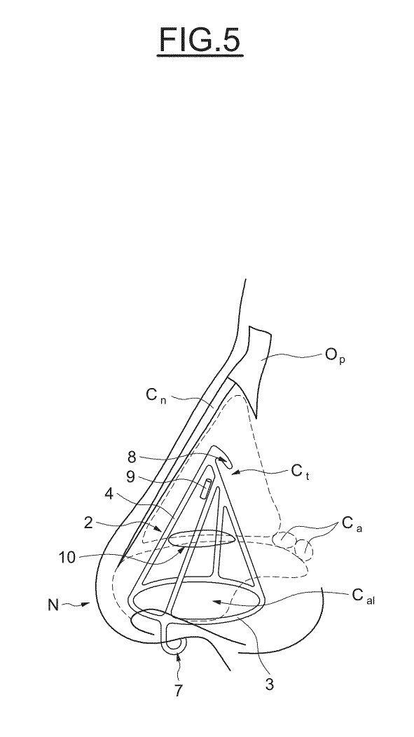

FIG. 5 shows a side view of another example of implementation of a device according to the invention, installed in a left nostril.

One will refer first of all to FIG. 1 which illustrates the basic structure of an ambulatory nasal device in conformity with the invention and to FIGS. 2 and 3 which show an example of implementation of an ambulatory nasal device inserted in a nostril NR of a user.

As it appears, the device comprises, in the embodiment represented on these figures, two structures of expansion intra-nasal (2) intended each one to be inserted in a nostril of a user.

The dimension of the structures of expansion is an anatomical standard and must answer two sizes:

“small” and “medium”.

Each intra-nasal structure of expansion has an overall pyramidal form and comprises an annular base (3), an opened base, and one or more arches (4) of triangular general form extending from the annular base.

The intra-nasal structure of expansion further comprises one or two side branches (5 and 6) whose ends are connected to the arch (4) and the annular base (3). The branches are joined together here in the vicinity of their end by which they are connected to the arch (4) so that these branches form a secondary arch.

The intra-nasal structures of expansion are connected by a spacer (7) punt, curved and discrete at the base of the nose. As indicated previously, and referring to FIGS. 2 and 3 on which one recognizes the bones of the nose (Op), the nasal partition (Cn), the triangular cartilage (Ct), the additional cartilages (tac) and the wing cartilage (Cal), the intra-nasal structures of expansion are intended to be inserted in the nostrils of a user. The surface of the base (3) of each structure of expansion is higher in the light of the nostril to ensure their in situ maintenance. The intra-nasal structures of expansion exert actually a support on the nasal mucous membrane over the entire length of the annular base, the arch (4) and the side branches (5 and 6) to support the opening of the nostril, while avoiding the blocking of secretions.

The structures of expansion can in addition be equipped with one or more digital-shaped extensions of variable form, plane or curved, judiciously placed to put targeted muco-cartilaginous pressure.

Dimensions of the extensions are related to the anatomy which they meet.

In this respect, the structures of expansion are equipped here with an end spacer (8) provided at the top of the arch (4) and including at least a digital-shaped appendix made in the form of a bent finger intended to be positioned and to exert a support on the area of the triangular cartilage to separate it.

Such a spacer remains however optional and could consequently be omitted.

In the embodiment of FIGS. 2 and 3, each structure of expansion can further be equipped internally with a tank (9) intended to receive a therapeutic substance. Such a tank can for example be fixed on the inside of the structure of expansion, for example on the arch or the side branches so as to be located along of the flow of air while remaining distant from the mucous membranes. Such a tank can have a slope, a site or a different form according to its function, for example diffusion, various therapies, etc. . . .

The assembly of elements can be made in only one operation of injection molding, which gives a perfect homogeneity to the whole of the structure. One will preferably realize the device by injection molding of a material with memory of form suited to exert efforts on the muco-cartilaginous zones on which it rests and presenting physical properties, in particular an elasticity, selected so as to regulate the effort which it exerts on these zones.

It will be noted in addition that, as indicated previously, each structure of expansion has a structure elastically deformable in particular thanks to its realization in the form of a pyramidal structure including an annular base and an arch extending from the annular base.

Moreover, as visible by the arrows (F) on FIG. 4, the air sucked up through the annular base (3) circulates up to the opposite end through the structure of expansion to follow its way towards the nasal fossae.

The device according to the invention is particularly intended for medico-surgical industry and the therapy concerning the oto rhyno laryngologic sphere.

In various applications, it can be used to provide a function of spacer and to present particular orientations adapted to the space met, according to the organe to be rectified, in order to get an effective solution with the respiratory embarrassment felt by a user, resulting from an architectural or mucous obstacle in the way of air flows.

Thus, the ambulatory nasal device initially provides a respiratory assistance thanks to the presence of the structures of expansion fitted in the nostrils and facilitating ventilation thanks to the opening of the respiratory tracts improving volume of air inspired or expired.

Such a device also makes it possible to get a certain number of therapeutic effects.

Initially, the structure of expansion provides a function of osseous, cartilaginous or mucous guidance in so far as, for example, the maintenance of a cartilage in a position, under the action of an effort exerted by the structure, can have a durable effect.

It will be noted nevertheless that, in addition to the therapeutic effects, thanks to its function of osseous, cartilaginous or mucous guidance, the structure of expansion can also be used for esthetics.

As indicated previously, the structure of expansion can also act as a spacer on the level of anatomical obstacles towards the nasal fossae.

When the function of guidance or spacer is required, the nasal device makes it possible to obtain a durable stability of the muco-cartilaginous site in which it fits, and is particularly effective for the reduction of architectural or mucous anatomical defects of the nasal valve and the nasal partition.

It has been noted that such a device is perfectly effective to adapt and request with spacing the triangular cartilage and the wing cartilage in the event of deficiency of the latter.

The device according to the invention can thus find a particularly interesting application to correct the anomalies of the triangular cartilage responsible for a loss of the integrity of the nasal valve and for a consecutive disturbance of ventilatory flow and to enable him to find its function of origin, while fitting between the triangular cartilage and the wing cartilage.

Thus, the ambulatory nasal device according to the invention aims initially at providing a respiratory assistance to a user. It can of course be a question of private use to provide a night autonomous respiratory assistance to users.

It can however also be a question of providing a post-operative respiratory assistance, in particular during the waking up of a patient, when the respiratory valves prevent him from breathing. The device can be the subject, in this case, of a manufacturing out of resorbable material.

In addition, thanks to its function of osseous guidance, cartilaginous or mucous and of spacing of anatomical obstacles towards the nasal fossae, the device can provide an alternative to surgical solutions dedicated to the treatment of obstructive pathologies reducing ventilation, by providing an orthopedic solution able to durably treat an obstructive anatomical configuration of the nasal tract and/or the nasal partitions.

Second, as indicated previously, the inside of the intra-nasal structure of expansion can provide a tank allowing the diffusion of effective therapeutic substances for the treatment of pathologies of nasal obstruction and more generally of the conduction of air in the higher air routes. Such can be in particular the case for in situ diffusion of therapeutic substances acting on problems of swelling of mucous shutters, for example of the horns.

It can also be a question of providing an accompaniment to the healing within the framework of a nasal plastic surgery or assistance to the hemostasis.

In addition, thanks to the presence of diffusion reserves of therapeutic substances, such a nasal device can find application to the assistance with nicotinic weaning or any other type of application requiring the diffusion of a therapeutic substance per nasal way.

It will be noted finally that the invention is not limited to the embodiment described in reference to FIGS. 1 to 3.

Indeed, in the embodiment described previously, the intra-nasal structure of expansion comprises only one ring forming the base of the pyramidal structure and an arch extending starting from the ring.

It would be of course possible, in alternative to equip the intra-nasal structure of expansion with one or several intermediate rings connected to the main arch and laid out for example between the top of the main arch and the annular base.

In the same way, while referring to FIG. 5, each intra-nasal structure of expansion can be equipped with a transverse partition schematically represented.

Such a partition can, for example, comprise a full wall, in this case, to form an obturator of nostrils which can be used, for example, by swimmers.

Such a partition (10) can also adopt a perforated structure or, in general, be made out of a material permeable to air so as to constitute a filter so that the device can advantageously be used in a polluted environment.

1. Ambulatory nasal device for respiratory assistance, including at least an intra-nasal structure (2) of expansion intended to be inserted in a nostril, characterized in that the structure of expansion presents a pyramidal form with an annular base (3) and comprises at least an arch extending from the afore mentioned base.

2. Device according to claim 1, characterized in that it is at least partly made out of a material elastically deformable with memory of form.

3. Device according to one of the claims 1 and 2, characterized in that each structure (2) of expansion comprises a main arch (4) of triangular general form, a main ring (3) connected to the base of the main arch and at least a branch (5, 6) connected to the main arch and said ring (4).

4. Device according to claim 3, characterized in that each structure of expansion comprises at least an intermediate ring connected to the main arch and laid out between the top of the main arch and the annular base.

5. Device according to any of claims 1 to 4, characterized in that the top of the structure of expansion comprises an end spacer including at least a digital-shaped appendix (8) intended to be positioned in the area of the triangular cartilage.

6. Device according to any of claims 1 to 5, characterized in that it comprises at least an inboard tank (9).

7. Device according to any of claims 1 to 6, characterized in that it comprises a pair of intra-nasal structures of expansion connected by a spacer (7).

8. Device according to any of claims 1 to 7, characterized in that each intra-nasal structure of expansion comprises a transverse wall (10) located on the way of airflow.

9. Device according to claim 8, characterized in that the transverse wall (10) is solid or perforated.

10. Device according to one from the claims 8 and 9, characterized in that the transverse wall (10) is made from a material permeable to the air.

11. Device according to any of claims 1 to 10, characterized in that it is made with resorbable material.
